(19) United States
(12) Patent Application Publication
(10) Pub. No.: US 2008/0227995 A1
(43) Pub. Date: Sep. 18, 2008

(54) Process of Sulfonating 4-Aminobenzonitriles

(75) Inventors: Jorg Hamann, Cologne (DE); Ralf Kanzler, Leverkusen (DE); Youxin Li, Langenfeld (DE)

Correspondence Address: EDWARDS ANGELL PALMER & DODGE LLP, P.O. BOX 55874, BOSTON, MA, 02205, US

(73) Assignee: Schwarz Pharma AG, Monheim Am Rhein (DE)

(21) Appl. No.: 11/989,082

(22) PCT Filed: Jul. 20, 2006
(86) PCT No.: PCT/EP2006/007170

§ 371 Date: Jan. 18, 2008

(30) Foreign Application Priority Data
Jul. 20, 2005 (EP) 05 015 790.8

(51) Int. Cl. C07C 331/24; C07C 303/36; C07C 311/08; C07C 303/40; C07C 335/12

(52) U.S. Cl. 558/17; 558/413; 560/24; 564/25; 558/18

(57) ABSTRACT

A process of producing a compound of the following formula (3):

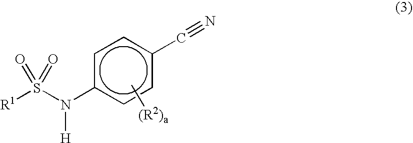

wherein
R¹ is a C₁₋₅ alkyl group,
R² is a halogen atom, a C₁₋₅ alkyl group, a C₂₋₅ alkenyl group, a C₂₋₅ alkynyl group, a C₁₋₅ alkyl group, a C₁₋₅ alkoxy group, a nitro group, or a hydroxy group, wherein multiple R² may be the same or may be different, and
a is an integer of from 0 to 4,
   comprising reacting a compound of the following formula (2):

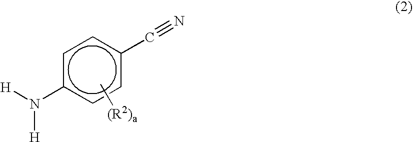

wherein R² and a are as defined above with a C₁₋₅-alkanesulfonyl chloride or C₁₋₅-alkanesulfonic acid anhydride followed by hydrolyzing an N,N-disulfonated derivative of compound (3) to the compound of formula (3).

### FIELD OF THE INVENTION

The present invention relates to a process of sulfonating 4-aminobenzonitriles. The present invention also relates to a process of producing vanilloid receptor antagonists. Specifically, the invention relates to a process of producing N-(alkylbenzyl)-N′-[4-(alkanesulfonylamino)-benzyl]urea and derivatives thereof. The invention further relates to processes of producing intermediates for the synthesis of vanilloid receptor antagonists such as N-(alkylbenzyl)-N′-[4-(alkanesulfonylamino)benzyl]thiourea and derivatives thereof.

### BACKGROUND

Recently, vanilloid receptor antagonist have attracted the attention of medicinal chemists and pharmacologist because of their potential use as drugs for treating pain, inflammatory diseases, ulcerous conditions etc. (Szallasi, J Med Chem 2004, 47, 2717; Tafesse, BMCL 2004, 14, 5513; Holzer, Eur J Pharmacol 2004, 500, 231; Wang et al, Mol Pharmacol 2002, 62, 947; Suh, BMCL 2003, 13, 4389; Doherty et al, J Med Chem 2005, 48, 71; WO 02/16318; WO 2005/003084; WO 2006/51378).

N-(4-t-butylbenzyl)-N′-[3-fluoro-4-(methanesulfonylamino)benzyl]thiourea gin the following referred to as SPM 14221) is an example of a potent vanilloid receptor antagonist (Wang et al., Mol Pharmacol 2002, 62, 947; Suh, BMCL 2003, 13, 4389; WO 02/16318) and is thus a valuable candidate for clinical development. However, the synthesis of vanilloid receptor antagonist such as SPM 14221 as described in the prior art has several drawbacks.

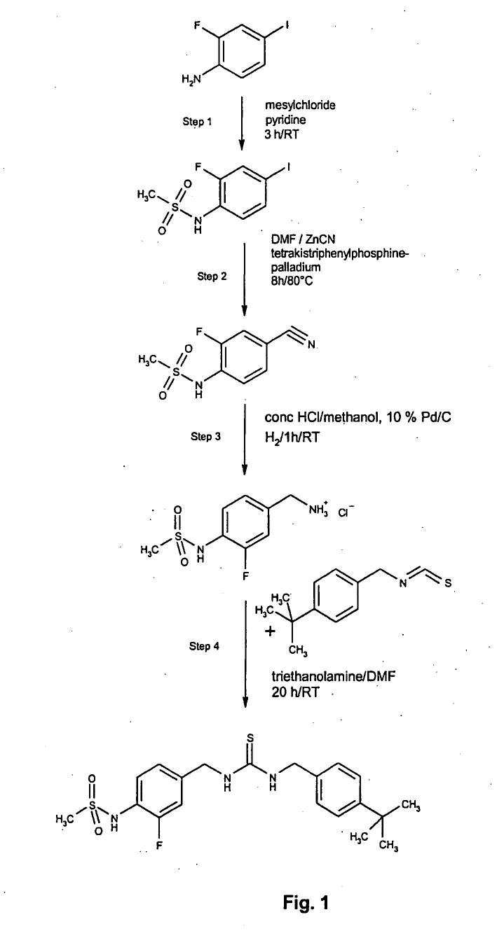

Using SPM 14221 as an example, a method of producing vanilloid receptor antagonists in the prior art (e.g. WO 02/16318) is shown in FIG. 1. The method starts out with 2-fluoro-4-iodoaniline and is performed according to the following steps, wherein steps 2a and 2b as well as steps 3a and 3b are alternative routes:
(1) methanesulfonyl chloride is added dropwise to 2-fluoro-4-iodoaniline and the reaction is allowed to proceed for 3 hrs. The mixture is then diluted with water and extracted with ethylacetate several times. The combined organic layers are washed, dried and concentrated, and N-(2-fluoro-4-iodophenyl)methanesulfonamide is then purified by chromatography.
(2) (a) zinc cyanide is added to N-(2-fluoro-4-iodophenyl)methanesulfonamide in the presence of a palladium catalyst and the mixture is heated at BOC for 8 hrs. The mixture is then diluted with water and extracted with ethyl acetate several times and the resulting N-(2-fluoro-4-cyanophenyl)methane-sulfonamide) is purified by column chromatography (b) in an alternative approach cupper cyanide is added to N-(2-fluoro-4-iodophenyl)methanesulfonamide and the mixture is heated to 130° C. (Suh et al, supra)
(3) (a) N-(2-fluoro-4-cyanophenyl)methanesulfonamide) is hydrogenated for 16 hrs in the presence of 10% palladium on carbon and concentrated hydrochloric acid to afford 3-fluoro-4-(methanesulfonyiamino)benzyl amine salt (b) in an alternative approach N-(2-fluoro-4-cyanophenyl)methanesulfonamide) is hydrogenated with BH₃ in THF. The mixture is then refluxed and treated with concentrated HCl (Suh et al, supra)
(4) 3-fluoro-4-(methanesulfonylamino)benzyl amine salt is then reacted with 4-tert-butylbenzyl isothiocyanate in the presence of triethylamine for 20 hrs. The mixture is then diluted with water and extracted with ethyl acetate several times and SPM 14221 is then purified by chromatography.

However, these methods are not suitable for the production of vanilloid receptor antagonists on a commercial scale. Particularly, prior art steps 1 and 2 (FIG. 1) are cumbersome and impractical on an industrial scale because they require several steps of dilution and solvent extraction and each step demands a final purification step using column chromatography.

Prior art step 1 further suffers from the high reactivity and therefore low selectivity of mesyl chloride or other alkanesulfonyl chlorides. Mesyl chloride reacts fast with humidity in air to methane sulfonic acid and gaseous HCl. Mesyl chloride is therefore difficult to apportion exactly, since it contains varying amounts of methane sulfonic acid that does not give the desired reaction product with 2-fluoro-4-iodoaniline. Moreover, the gaseous reaction product HCl presses mesyl chloride out of many instruments normally used for exact apportionment such as pipettes. As a result, it is very difficult to add exactly one molar equivalent of mesyl chloride to a given amount of a substrate. If less than one molar equivalent of mesyl chloride is used, the yield of the desired product is insufficient. Therefore, a small molar excess of mesyl chloride is typically used in the prior art. An excess of mesyl chloride, however, leads, due to the low selectivity of mesyl chloride, to disulfonated products, also decreasing the yield of the desired monosulfonated product. Further, additional purification steps such as column chromatography are necessary for removing the disulfonated product and other impurities formed from 2-fluoro-4-iodoaniline under the harsh conditions of excessive mesyl chloride. Even if one manages to apportion exactly one equivalent of mesyl chloride to 2-fluoro-4-iodoaniline, it is difficult to exclude formation of the disulfonated product. High volumes of dry solvent and very slow addition of mesyl chloride are then necessary to suppress the formation of undesired products.

It is therefore an object of the present invention to overcome the problems associated with the prior art and to provide a simple, safe and economical process of producing monosulfonated 4-aminobenzonitriles and derivatives thereof with high yield and high purity. It is another object of the invention to provide a simple, safe and economical process of producing vanilloid receptor antagonists such as N-(alkylbenzyl)-N′-[4-(alkanesulfonyl-amino)benzyl]thiourea compounds. These processes should be suitable for upscaling to commercial scale and should provide the desired thiourea, urea or amide compounds in high yield and purity. It is a further object of the invention to provide a simple process of producing benzyl isothiocyanates suitable for the production of said thiourea compounds. It is a further object of the invention to provide benzyl thiocyanates.

### GENERAL DESCRIPTION OF THE INVENTION

The above objects have been solved by the present invention. The invention provides a process of producing a compound of the following formula (3):

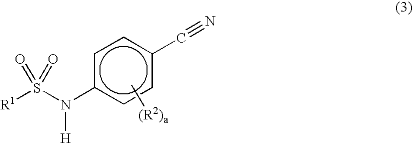

wherein

R¹ is a C₁₋₅ alkyl group,

R² is a halogen atom, a C₁₋₅ alkyl group, a C₂₋₅ alkenyl group, a C₂₋₅ alkynyl group, a halo C₁₋₅ alkyl group, a nitro group, a hydroxy group, or a C₁₋₅ alkoxy group, wherein multiple R² may be the same or may be different, and

a is an integer of from 0 to 4,

comprising reacting a compound of the following formula (2):

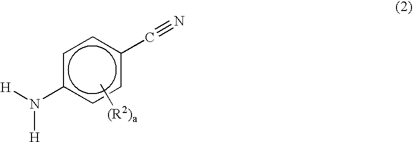

wherein R² and a are as defined above with a C₁₋₅-alkanesulfonyl halide, preferably a C₁₋₅-alkanesulfonyl chloride, or C₁₋₅-alkanesulfonic acid anhydride as sulfonating agent, followed by hydrolyzing an N,N-disulfonated derivative of compound (3) to the compound of formula (3) in an aqueous solvent. In one embodiment, the compound of formula (2) is treated with more than one molar equivalent of C₁₋₅-alkanesulfonyl halide or C₁₋₅-alkanesulfonic acid anhydride to produce a reaction mixture containing a disulfonated product of the following formula (3a):

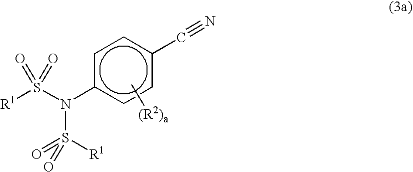

followed by hydrolyzing the compound of formula (3a) to a compound of formula (3) in an aqueous solvent.

The invention further provides a process of producing a compound of the following formula (1):

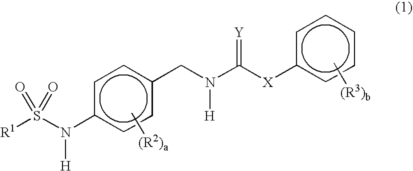

wherein
X is —NH—CH₂—, —CH₂—CH₂—, —CH═CH—, or —C≡C—,
Y is O or S,
R¹ is a C₁₋₅ alkyl group,
R² is a halogen atom, a C₁₋₅ alkyl group, a C₂₋₅ alkenyl group, a C₂₋₅ alkynyl group, a halo C₁₋₅ alkyl group, a nitro group, a hydroxy group, or a C₁₋₅ alkoxy group, wherein multiple R² may be the same or may be different, and
R³ is a halogen atom, a C₁₋₆ alkyl group, a C₂₋₅ alkenyl group, a C₂₋₅ alkynyl group, a halo C₁₋₆ alkyl group, a C₁₋₄ alkoxy group, a C₁₋₅ alkylthio group, a nitro group, a C₁₋₅ alkoxy C₁₋₅ alkoxy group, a C₁₋₅ alkoxy C₁₋₅ alkyl group, a C₁₋₅ alkoxy C₁₋₅ alkoxy C₁₋₅ alkyl group, C₁₋₅ alkylsulfonyl group, C₁₋₅ alkylcarbonyl group, C₁₋₅ alkoxycarbonyl group, C₁₋₅ alkoxycarbonyl C₁₋₅ alkoxy group, a C₁₋₅ alkoxy C₁₋₅ alkylamino group, morpholino, wherein multiple R³ may be the same or may be different
a is an integer of from 0 to 4, and
b is an integer of from 0 to 5,

comprising the following step (i):
(i) converting a compound of the following formula (2)

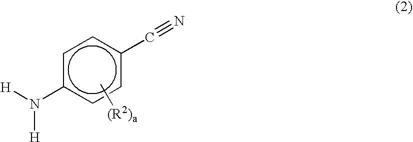

wherein R² and a are as described for formula (1) to a compound of the following formula (3):

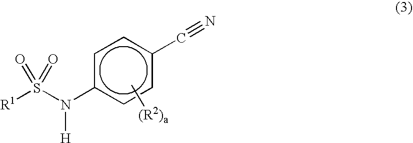

by reacting a compound of formula (2) with a C₁₋₅-alkanesulfonyl halide (such as a C₁₋₅-alkanesulfonyl chloride) or C₁₋₅-alkanesulfonic acid anhydride followed by hydrolyzing a disulfonated derivative of compound (3) to a compound of formula (3) in an aqueous solvent.

The invention further provides a process of producing a compound of the following formula (1-1):

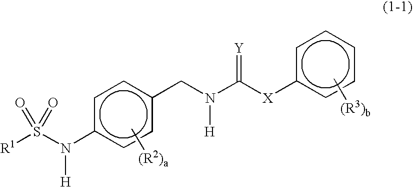

wherein
X is —NH—CH—, CH₂—, —CH₂—, —CH═CH—, —C≡C—, or —C(R⁴)₂—O—,
Y is O or S,
R¹ is a C₁₋₅ alkyl group,
R² is a halogen atom, a C₁₋₅ alkyl group, a nitro group, a hydroxy group, or a C₁₋₅ alkoxy group, wherein multiple R² may be the same or may be different, and
R³ is a halogen atom, a C₁₋₆ alkyl group, a C₂₋₅ alkenyl group, a C₂₋₅ alkynyl group, a halo C₁₋₆ alkyl group, a C₁₋₅ alkoxy group, a C₁₋₅ alkylthio group, a nitro group, a C₁₋₅ alkoxy C₁₋₅ alkoxy group, a C₁₋₅ alkoxy C₁₋₅ alkyl group, a C₁₋₅ alkoxy C₁₋₅ alkoxy C₁₋₅ alkyl group, C₁₋₅ alkylsulfonyl group, C₁₋₅ alkylcarbonyl group, C₁₋₅ alkoxycarbonyl group, C₁₋₅ alkoxycarbonyl C₁₋₅ alkoxy group, a C₁₋₅ alkoxy C₁₋₅ alkylamino group, morpholino, wherein multiple R³ may be the same or may be different
R⁴ is hydrogen, a C₁₋₅ alkyl group, or halogen, whereby multiple R⁴ may be the same or may be different,
a is an integer of from 0 to 4, and
b is an integer of from 0 to 5,

comprising the following step (i):
(i) converting a compound of the following formula (2)

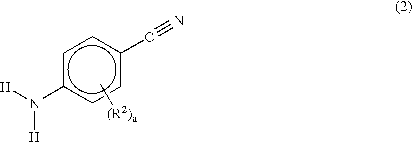

wherein R² and a are as defined for formula (1-1) to a compound of the following formula (3):

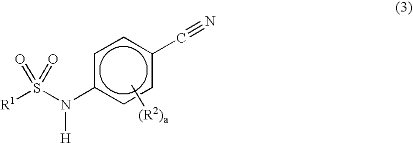

by reacting a compound of formula (2) with a C₁₋₅-alkanesulfonyl halide (such as a C₁₋₅-alkanesulfonyl chloride) or C₁₋₅-alkanesulfonic acid anhydride followed by hydrolyzing a disulfonated derivative of compound (3) to a compound of formula (3) in an aqueous solvent.

If X is —NH—CH₂—, the C atom of the —NH—CH₂— group is bonded to the benzene ring carrying the R³ group(s). If X is —C(R⁴)₂—O—, the O atom of the —C(R⁴)₂—O— group is bonded to the benzene ring carrying the R³ group(s). If X is —C(R⁴)₂—O—, Y is preferably O. If X is —CH═CH—, the compound of formula (1) may be the cis or the trans isomer.

In one embodiment of formula (3) or (1), R¹ is methyl or ethyl; R² is methyl, ethyl, vinyl, ethynyl, fluoro, chloro, bromo, iodo or nitro; a is 1 or 2. In one embodiment of formula (1-1), R¹ is methyl or ethyl; R² is methyl, ethyl, fluoro, chloro, bromo, iodo or nitro; a is 1 or 2.

If a is at least 1, at least one R² may be in ortho position to the position substituted by the amino or alkanesufonamido group. In another embodiment, R¹ is methyl or ethyl, R² is a fluorine or chlorine atom, a is 1 or 2, R³ is t-butyl or i-propyl, and b is 1. In a further embodiment, b is an integer of from 1 to 3 and at least one R³ is a branched C₁₋₆alkyl group or branched halo C₁₋₆-alkyl group in para position to group X. In a further embodiment, at least one R³ is an optionally halogenated t-butyl or i-propyl in para position to group X, whereby b may be an integer of from 1 to 3. In a further embodiment, at least one R³ in ortho or meta position to X is a halogen or a C₁₋₆ alkoxy group. In a further embodiment, Y is O. In another embodiment, at least one R⁴ is hydrogen.

In a further embodiment, Y is S, X is —NH—CH₂—, R² is a halogen atom or a C₁₋₅ alkyl group or a vinyl group, and R³ is a C₁₋₆ alkyl group or a halogen atom.

In one embodiment, step (i) is followed by the following step (ii):
(ii) converting a compound of formula (3) to a compound of the following formula (4) or a salt thereof:

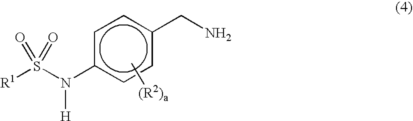

wherein R² and a are either as defined for formula (1) or as defined for formula (1-1).

In one embodiment of the above step (ii), R² may be a halogen atom, a C₁₋₅ alkyl group, or a C₁₋₅ alkoxy group.

In another embodiment, step (ii) is followed by the following step (iii-a):
(iii-a) converting a compound of formula (4) or the salt thereof with a compound of the following formula (5) to a compound of formula (1) or (1-1):

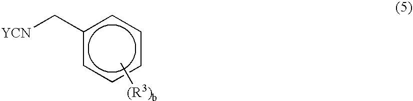

wherein Y, R³ and b are as defined above for formula (1).

In another embodiment, the process of producing a compound of formula (1) or (1-1) comprises the following step (iii-b):
(iii-b) converting a compound of formula (4) wherein R² and a are as defined for formula (1) or (1-1), respectively, or a salt thereof with a compound of the following formula (8) with a condensing agent to a compound of formula (1) or (1-1)

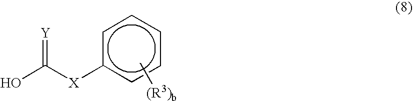

wherein Y, R³, and b are as defined for formula (1) and X is —CH₂—CH₂—, —CH═CH—, —C≡C—, or —C(R⁴)₂—O—.

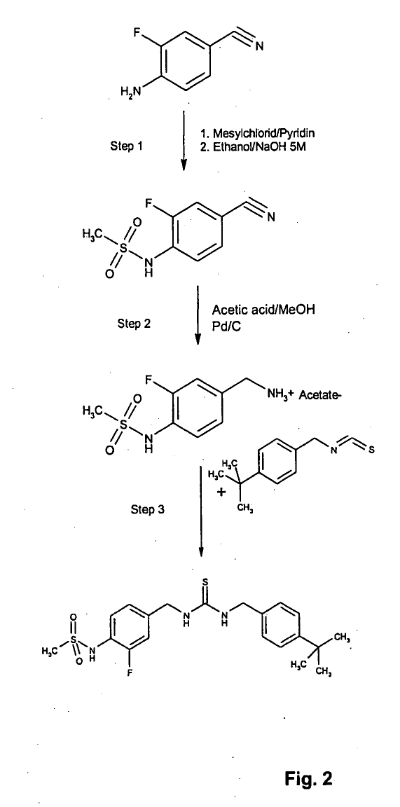

The inventors have surprisingly found that the process of producing the compound of formula (3) can be simplified by starting with the 4-aminobenzonitrile of formula (2) and, preferably, using the sulfonating agent in excess of the compound of formula (2). Any disulfonated reaction products of formula (3a) can be hydrolyzed thereafter to the monosulfonated compounds of formula (3). Surprisingly, the hydrolysis can be performed such that exclusively disulfonated products are hydrolyzed to the monosulfonated compounds without hydrolysis of monosulfonated compounds, whereby a reaction mixture essentially free of disulfonated products is obtained. As a result, the desired monosulfonated compound of formula (3) can in many cases be crystallized from the reaction mixture in high purity. Laborious workup using extraction and column chromatography may be performed if desired but is not necessary in many cases. The yield obtained in the method of the invention is very high, since essentially no disulfonated by-products remain after hydrolysis. Thus, the invention replaces a reaction that is difficult to control due to the high reactivity of the sulfonating agent by a two-step procedure, wherein the required selectivity for the monosulfonated compound is achieved not during sulfonation but during a subsequent hydrolysis step. The process of the invention is depicted in FIG. 2 using SPM 14221 as an example.

The invention further provides a process of producing a compound of formula (5) as defined above, said process comprising converting a compound of formula (6):

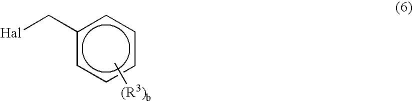

wherein HaI is a halogen atom and R³ and b are as defined above for formula (1) with rhodanide to a thiocyanate of the following formula (7):

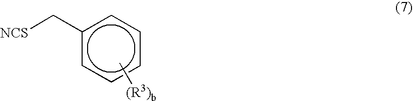

followed by converting the thiocyanate of formula (7) to a isothiocyanate of formula (5).

The invention further provides a process of producing a compound of formula (1) or (1-1), wherein Y is S and as further defined above, comprising the subsequent steps of
(a) converting a compound of formula (6) as defined above with rhodanide to the isothiocyanate of formula (5) and
(b) converting the isothiocyanate of formula (5) with a compound of formula (4) as defined above.

The invention further provides a process of producing a compound of formula (1) as defined above, comprising the reduction of a compound of formula (3) wherein R² and a are as defined for formula (1) to a compound of formula (4) or a salt thereof in acetic acid using palladium on carbon as a catalyst in the presence of hydrogen. The invention further provides a process of producing a compound of formula (1-1) as defined above, comprising the reduction of a compound of formula (3) wherein R² and a are as defined for formula (1-1) to a compound of formula (4) or a salt thereof as defined above in acetic acid using palladium on carbon as a catalyst in the presence of hydrogen.

The invention further provides a compound of the following formula (7):

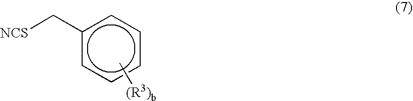

wherein R³ and b are as defined above. Preferably, R³ is a C₁₋₆ alkyl group and b is an integer of from 1 to 5, more preferably of from 1 to 3. Most preferably, b is 1 and R³ is in para position. R³ may for example be an optionally halogenated i-propyl or t-butyl.

The invention further provides the use of the compound of formula (7) in a method of producing a compound of formula (1) or of formula (1-1).

The invention further provides the use of a compound of formula (3) wherein R² and a are as defined for formula (1) or (1-1) for producing a compound of formula (1) or (1-1), respectively. Specifically, the invention provides the use of 3-fluoro-4-amino-benzonitrile in a method of producing N-(4-tert-butylbenzyl)-N′-[3-fluoro-4-(methanesulfonylamino)benzyl]thiourea.

FIG. 1 shows a prior art process of producing SPM 14221 (Wang et al., Mol. Pharm (2002)).

FIG. 2 shows the process of the invention using SPM 14221 as an example.

### DETAILED DESCRIPTION OF THE INVENTION

Herein, the radicals R¹, R², R³, and R⁴ may be any radicals as far as they are compatible with the processes of the invention. The preferred groups given below lead to vanilloid receptor antagonists of formula (1). However, the processes of the invention may be used for preparing compounds other than vanilloid receptor antagonists, whereby no limitations exist as to R¹, R², R³, and R⁴, as far as the processes of the invention are not compromized.

Herein, the halogen atom may be a fluorine, chlorine, bromine, or iodine atom. The terms “halo” and “halogen atom” as substituents are used exchangeably herein. The C₁₋₅ alkyl group may be a linear, branched or cyclic C₁₋₅ alkyl group, examples of which are methyl, ethyl, n-propyl, i-propyl, n-butyl, i-butyl, t-butyl, pentyl, cyclopropyl, cyclobutyl, cyclopentyl etc. The C₁₋₆ alkyl may be, in addition to the examples given for the C₁₋₅ alkyl group, a linear, branched or cyclic hexyl group. The halo C₁₋₆ alkyl group is a C₁₋₆ alkyl wherein one or more hydrogen atoms of the C₁₋₆ alkyl group are substituted by a halogen atom.

The C₁₋₅ alkyl group of R¹ is preferably methyl or ethyl, and a methyl group is most preferred.

The C₂₋₅ alkenyl group may be a linear or branched C₂₋₅ alkenyl group such as vinyl, n-propenyl (—CH₂CH═CH₂), isopropenyl (—C(CH₃)═CH₂), butenyl etc.

R² is as defined for formula (1) or formula (1-1). In another embodiment, R² is a halogen atom or a C₁₋₅ alkyl group. If R² is a C₁₋₅ alkyl group, a methyl or ethyl group is preferred. If R² is a halogen atom, fluorine or chlorine are preferred, and fluorine is most preferred.

Index a indicates the number of groups R² on the phenyl group to which R² may be attached a is an integer of from 0 to 4. In one embodiment, a is an integer of from 0 to 2. In another embodiment, a is 1. If a is 1, it may be located in ortho or meta position to the sulfonated amino group, whereby the ortho position is preferred.

R³ in ortho, meta or para position to X are independently a halogen atom, a C₁₋₆ alkyl group, a C₂₋₅ alkenyl group, a C₂₋₅ alkynyl group, a halo C₁₋₆ alkyl group, a C₁₋₅ alkoxy group, a C₁₋₅ alkylthio group, a nitro group, a C₁₋₅ alkoxy C₁₋₅ alkoxy group, a C₁₋₅ alkoxy C₁₋₅ alkyl group, a C₁₋₅ alkoxy C₁₋₅ alkoxy C₁₋₅ alkyl group, C₁₋₅ alkylsulfonyl group, C₁₋₅ alkylcarbonyl group, C₁₋₅ alkoxycarbonyl group, C₁₋₅ alkoxycarbonyl C₁₋₅ alkoxy group, a C₁₋₅ alkoxy C₁₋₅ alkylamino group, or morpholino, wherein multiple R³ may be the same or may be different. Positions not substituted by any of these groups are occupied by hydrogen atoms.

In one embodiment, a group R³ in para position to X is a C₃₋₆ alkyl group or a halo C₃₋₆ alkyl group, whereby branched groups such as i-propyl and t-butyl or halogenated derivatives thereof are preferred. A t-butyl group in para position to X is most preferred.

In another embodiment, R³ in ortho or meta position to group X is a halogen atom, a C₁₋₅ alkoxy or a C₁₋₅ alkoxy C₁₋₅ alkoxy group.

Index b indicates the number of groups R³ on the phenyl group to which R³ may be attached. b is an integer of from 0 to 5, preferably an integer of from 1 to 3, and most preferably 1. If b is 1, R³ may be located in ortho, meta or para position to group X attached to the ring to which R³ may be attached, whereby the para position is preferred. If b is greater than 1, it is preferred that one R³ is in para position to group X. In para position to X, a branched alkyl or haloalkyl group is preferred as R³.

HaI is a halogen atom that is preferably chlorine or bromine, most preferably bromine.

C₁₋₅-alkanesulfonyl halide and C₁₋₅-alkanesulfonic acid anhydride are referred to herein as “sulfonating agent”. Regarding the C₁₋₅-alkane group of these sulfonating agents, the definitions given above for R¹ apply. With regard to halide group of the C₁₋₅-alkanesulfonyl halide, chloride and bromide are preferred and chloride is most preferred.

The salts of the compound of formula (1) or (4) are not particularly limited. Said salt may be a salt of an organic or an inorganic acid, e.g. formate, acetate, citrate, tartrate, maleate, malate, succinate, hydrochloride, sulfate, hydrogensulfate etc. Preferred salts are acetate and hydrochloride, most preferred is acetate.

Possible embodiments of formula (1) or formula (1-1) with respect to R¹, R², and R³, a and b are as follows:

R¹ is a C₁₋₅ alkyl group, R² is a halogen atom or a C₁₋₅ alkyl group, a is an integer of from 0 to 4, R³ is a C₁₋₆ alkyl group or halo C₁₋₆ alkyl group, and b is an integer of from 0 to 5;

R¹ is a C₁₋₅ alkyl group, R² is a halogen atom or a C₁₋₅ alkyl group, a is an integer of from 1 to 4, R³ is a C₁₋₆ alkyl group, and b is an integer of from 1 to 5;

R¹ is a methyl group, R² is a halogen atom or a C₁₋₅ alkyl group, a is an integer of from 1 to 4, R³ is a C₁₋₆ alkyl group, and b is an integer of from 1 to 5;

R¹ is methyl or ethyl, R² is a fluorine atom, chlorine atom, methyl or ethyl, a is 1 or 2, R³ is t-butyl, i-propyl, chlorine or bromine, and b is 1 or 2;

R¹ is methyl or ethyl, R² is a fluorine atom, chlorine atom, methyl or ethyl, a is 1 or 2, R³ is t-butyl or i-propyl, and b is 1 or 2;

R¹ is methyl, R² is fluorine atom or chlorine atom, a is 1 or 2, R³ is t-butyl or i-propyl in para position, and b is 1;

R¹ is methyl or ethyl, R² is fluoro, and a is 1 or 2;

if a is 1 or higher, at least one of R² is preferably located in ortho position with respect to the amino group of formula (2) or the sulonated amino group of formula (1) and (3); and preferably one R³ is in para position to group X.

In any of these embodiments, Y may be O or S, whereby Y is preferably O. Preferred embodiments for X in any of these embodiments are —NH—CH₂— and —CH═CH—.

As condensing agent, any of those listed on page 14 of WO 2006/51378 as coupling agents may be used. Preferred condensing agents are DCC and EDC.

Next, the invention is described with reference to preferred compounds, compound classes, or reactions.

The process of producing the compound of formula (1), notably SPM 14221, comprises the process of producing the compound of formula (3). The compound of formula (2) is first reacted with a sulfonating agent such as methane sulfonic acid chloride (mesyl chloride) or methane sulfonic acid anhydride, preferably in the presence of an organic base, followed by hydrolyzing any N,N-disulfonated intermediate of formula (3a) such as N-(4-cyano-2-fluoro-phenyl)-N-methanesulfonyl-methanesulfonylamide, if present, in an aqueous solvent with a base such as alkali to the compound of formula (3) such as N-(2-fluoro-4-cyanophenyl)methansulfonamide, which is exemplified by the following scheme:

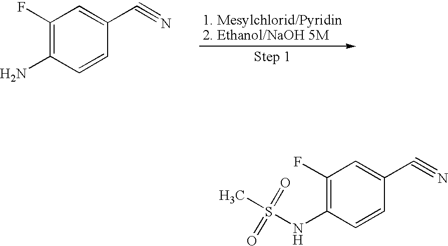

This process can be performed easily and in good yield (usually over 90% of Th.) and the product of formula (3) can usually be obtained in a purity of about 99%, whereby additional purification steps are frequently not necessary. However, if desired, it is also possible to further purify the compound of formula (3) by conventional methods such as recrystallization or chromatography. The process of the invention avoids the use of metallocyanides and. palladium catalysts as described in the prior art and can be easily upscaled in the kg range as illustrated in Example 3.

The sulfonation of the compound of formula (2) such as 3-fluoro-4-amino-benzonitrile with the sulfonating agent such as mesyl chloride or methanesulfonic acid anhydride should be performed in the presence of an organic base such as a tertiary alkylamine, an N-substituted morpholine or pyridine, wherein pyridine is particularly preferred.

The sulfonation can be performed at 0-50° C. for 2.5-5 hrs and preferably at 20-25° C. for about 3 hrs. At least 1 molar equivalent of sulfonating agent with respect to the amount of the compound of formula (2) should be used. Preferably, at least 1.2 molar equivalents, more preferably at least 1.5 molar equivalents, more preferably at least 2.0 and most preferably about 2.5 molar equivalents of sulfonating agent are used;

The sulfonation usually leads to disulfonated products of formula (3a) such as N-(4-cyano-2-fluoro-phenyl)-N-methanesulfonyl-methanesulfonylamide in varying amounts even if no or only a slight-excess of sulfonating agent is used. The amount of the disulfonated product obtained depends inter alia on the excess of the sulfonating agent, on the amount of solvent used and on the speed at which the sulfonating agent is added to the compound of formula (2). However, in the prior art processes, it is difficult to avoid formation of disulfonated products completely. The invention provides a selective hydrolysis step that converts any disulfonated product of formula (3a) to the monosulfonated product of formula (3).

The hydrolysis step of the invention can be performed by heating the disulfonated compound of formula (3a) or a mixture of the disulfonated compound of formula (3a) and the monosulfonated compound of formula (3) in an aqueous solvent in the presence of a base. Preferably, the base is a strong organic or an inorganic base such as NaOH, KOH or aqueous amines such as pyridine/water. These bases are preferably added to the reaction mixture of the sulfonation reaction to give the concentrations of base given below, followed by heating. Under the conditions given in the following, selective hydrolysis to the monosulfonated compounds of formula (3) is achieved.

The concentration of the base may be at least 2 M, preferably at least 2.5 M and most preferably at least 3M. The concentration of the base may be in the range of from 3 to 6 M, preferably from 3 to 4 M. The reaction may be performed at temperatures elevated above room temperature, such as a temperature of from 30° C. to reflux temperature, preferably 50 to 100° C. and most preferably between 80 to 100° C. The reaction may be conducted for 0.5-3 hrs in an appropriate aqueous solvent system such as THF, acetone or alcohols in the case of NaOH or KOH as a base. If pyridine is used as the base, preferably 12 molar equivalents pyridine (based on the amount of the compound of formula (2)) are used with the double amount (vol/vol) of water and then the mixture may be stirred for 45-90 Minutes at 90-100° C.

Advantageously, the hydrolysis step is performed in the same vessel as used for the sulfonation reaction by adding further base as required and by heating the vessel to the temperature required for hydrolysis for the required period of time.

Preferably, the same base is used during hydrolysis as is used for sulfonation. In this case, it may be sufficient to dilute the reaction mixture of the sulfonation step with water to achieve an aqueous solution of the base (see example 1). In one embodiment, pyridine is used as a base for this purpose.

The compound of formula (3) may be crystallized from the reaction mixture obtained from hydrolysis by cooling e.g. to 0° C. It may be isolated in high purity by filtration. Further, purification steps such as column chromatography are usually not required.

The compound of formula (3) such as N-(2-fluoro-4-cyanophenyl)methanesulfonamide can then be used to produce a compound of formula (1) such as SPM 14221 as described in the prior art.

The present invention provides improvements of the subsequent steps of the synthesis of compounds of formula (1).

Reduction step 3(a) of the prior art process includes the use of concentrated hydrochloric acid to produce 3-fluoro-4-(methanesulfonylamino)benzyl amine salt. However, concentrated hydrochloric acid attacks common autoclaves and is impractical to handle on an industrial scale. Also, the large amount (50%) of palladium on carbon catalyst used in prior art is expensive.

Suh et al. (2003, supra) therefore proposed an alternative method of reducing N-(2-fluoro-4-cyanophenyl)methanesulfonamide using BH₃. However, BH₃ is expensive and the use of concentrated hydrochloric acid on an industrial scale should be avoided for economical and ecological reasons. It was hence an object of the invention to provide an alternative reduction step which eliminates the use of BH₃ and concentrated hydrochloric acid. This object has been solved by a process using about 5 wt % palladium/carbon catalyst (based on the amount of the compound of formula (3)) in the presence of 2-5 molar equivalents acetic acid, preferably 3 to 3.5 molar equivalents acetic acid (based on the amount of the compound of formula (3)). The reduction may be performed at a temperature of between 7 and 14° C. The solvent may be a C₁₋₃ alkanol such as methanol. The reaction is exemplified by the following scheme.

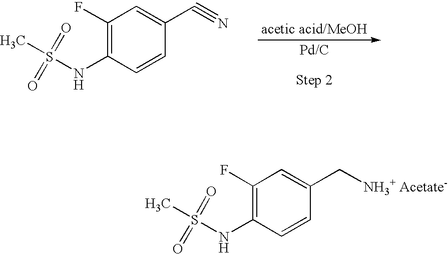

This reaction can be performed with good yield (>85%) and excellent purity (>99%) of the compound of formula (4) or the salt thereof, such as of 3-fluoro-4-(methanesulfonylamino)-benzyl amine salt.

Accordingly, one embodiment of the present invention is a process of producing a compound of formula (1) or (1-1), comprising the reduction of a compound of formula (3) wherein R² and a are as defined for formula (1) or (1-1), respectively, such as of N-(2-fluoro-4-cyanophenyl)methanesulfonamide, to a compound of formula (4) or a salt thereof, such as 3-fluoro-4-(methanesulfonylamino)benzyl amine salt, in acetic acid using palladium on carbon, preferably using at most 5 wt % palladium/carbon as a catalyst.

Alternatively, the reduction of a compound of formula (3) such as N-(2-fluoro-4-cyanophenyl)methanesulfonamide to the compound of formula (4) or a salt thereof may be done using Raney nickel as a catalyst. The reaction can be performed using a C₁₋₃ alkanol as the solvent system, wherein ethanol/NH₃ in water is preferred. The yield of this reaction typically exceeds 90% and the purity of the compound of formula (4) such as 3-fluoro-4-(methanesulfonylamino)-benzyl amine salt can be above 99%. However, the major impurity of this reaction is nickel which is brought into the product by the catalyst used. For this reason, the palladium/C catalyst reduction process as described above is preferred.

Alternatively, the reduction of the compound of formula (3) may be performed using lithium aluminium hydride as the reducing agent. The reaction can be performed by slowly adding 0.5-2 molar equivalents lithium aluminium hydride (based on the educt) to the compound of formula (3) such as N-(2-fluorocyanophenyl)methanesulfonamide (the educt) in anhydrous THF at a temperature of about 0-10° C. The mixtures may then be warmed up to room temperature or, preferably, to reflux for about 6 to 24 hrs, e.g. for 6 to 12 hrs. The reduction reaction can be stopped by adding concentrated (50%) NaOH or 1-5 N hydrochloric acid and after stirring for further 20-100 minutes, the precipitate can be washed and the product can be isolated.

The compound of formula (4) or the salt thereof, such as 3-fluoro-4-(methanesulfonylamino)benzyl amine salt, may then be converted with a compound of formula (5), such as 4-t-butylbenzyl isothiocyanate, to a compound of formula (1) or (1-1) (step iii), such as SPM 14221, as exemplified in the following scheme.

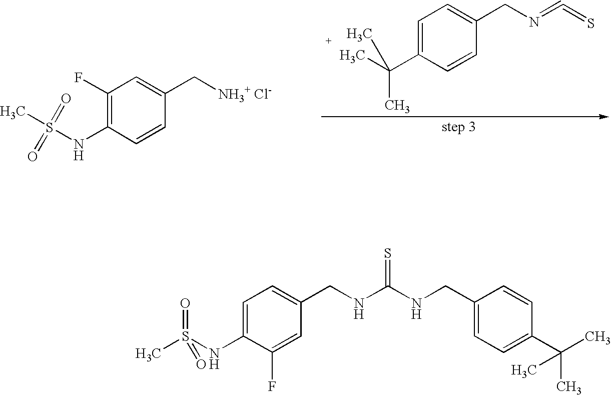

This step is analogous to that described in the prior art, wherein 4-t-butylbenzyl isothiocyanate is also used as the reagent. In the present invention, the reaction is optimized by using 5.2 molar equivalents triethylamine and by adding isothiocyanate in ethyl acetate solution. The reaction is preferably allowed to proceed for 1.5-2 hrs at 25° C. to 30° C. The final product is then recrystallized from methanol.

In the publications of Wang et al. and Suh (supra), no source for 4-t-butylbenzyl isothiocyanate is disclosed. According to WO 02/16318, 4-t-butylbenzyl isothiocyanate can be produced by adding thiophosgene to 4-t-butylbenzylamine. However, thiophosgene is toxic, badly smelling and its disposal is expensive and causes ecological problems.

It is thus another object of the invention to avoid the use of thiophosgene in the production of a compound of formula (5), such as 4-t-butylbenzyl isothiocyanate.

This object has been solved by a process of producing a compound of formula (5), comprising reacting a compound of formula (6), such as 4-t-butylbenzylbromide, with rhodanide, as illustrated by the following scheme:

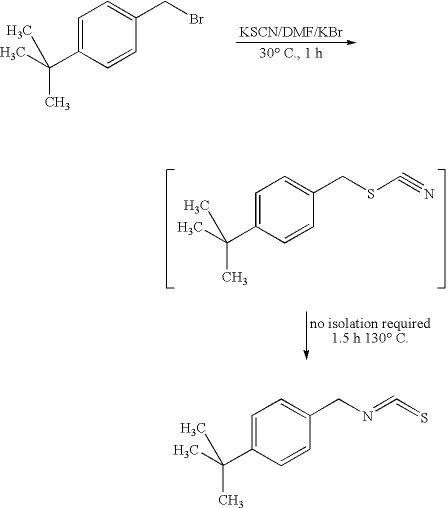

This reaction can be performed at 25-40° C. for 45-120 min. The reaction leads to a compound of formula (7) such as 1-t-butyl-4-thiocyanomethylbenzene as a stable intermediate which can be converted to a compound of formula (5) such as 4-t-butylbenzyl isothiocyanate by heating to 120-150° for 1-3 hours. In a convenient approach, both reactions can be performed without isolating the compound of formula (7) by heating the reaction mixture containing the compound of formula (6) and rhodanide to 120 to 150° C., preferably to about 130° C., for 1-4 hours. Said rhodanide may be an alkali metal rhodanide such as sodium or potassium rhodanide, whereby potassium rhodanide is preferred.

One aspect of the invention is thus a process of producing the compound of formula (5), such as 4-t-butylbenzyl isothiocyanate, by reacting a compound of formula (6), such as 4-t-butylbenzylbromide, with rhodanide, preferably with potassium rhodanide, to give a compound of formula (7), such as 1-t-butyl-4-thiocyanomethylbenzene, which may then be heated for 0.5-4 hours and preferably for 1-3 hrs to 120-150° C. to give a compound of formula (5), such as 4-t-butylbenzyl isothiocyanate. This reaction may be carried out in a polar solvent such as dimethyl formamide (DMF).

The conversion of a compound of formula (7) such as 1-t-butyl-4-thiocyanomethylbenzene to a compound of formula (5) such as 4-t-butylbenzyl isothiocyanate is preferably done in the presence of a catalyst. Common catalysts such as ZnCl₂ can be used fur this purpose. However, the inventors have surprisingly found that an inorganic bromide salt, such as KBr or NaBr can be also be used as a catalyst in this reaction.

Another aspect of the present invention is a process of producing a compound of formula (5), such as 4-t-butylbenzyl isothiocyanate, by reacting a compound of formula (6), such as 4-t-butylbenzylbromide, with rhodanide, preferably with potassium rhodanide; to a temperature of at least 120° C., preferably to 120-150° C., for about 1 to 4 hours.

Another aspect of the present invention is a method of producing SPM 14221 comprising the subsequent steps of

(a) reacting 4-t-butylbenzylbromide with a rhodanide to give 4-t-butylbenzyl isothiocyanate and

(b) reacting 3-fluoro-4-(methanesulfonylamino)benzyl amine salt with 4-t-butylbenzyl isothiocyanate to give SPM 14221.

1-t-butyl-4-thiocyanomethylbenzene is an important intermediate in the production of 4-t-butylbenzyl isothiocyanate and finally of SPM 14221. The compound has not been described before and represents a further aspect of the present invention.

A further aspect of the present invention is the use of 1-t-butyl-4-thiocyanomethylbenzene for the production of 4-t-butylbenzyl isothiocyanate. Another aspect of the present invention is the use of 1-t-butyl-4-thiocyanomethylbenzene in the production of SPM 14221.

Reactions to prepare compounds of formula (1) or (1-1) from respective compounds of formula (3) are known to the skilled person from the general prior art. In the following, guidance to these reactions is provided.

The urea and thiourea derivatives (wherein X is —NH—CH₂—) of the compounds of formula (1) or (1-1) may be prepared by reacting an amine of formula (4) wherein R² and a are as defined for formula (1) or (1-1), respectively, with a isothiocyanate or isocyanate of formula (5), respectively.

One embodiment of the present invention is thus a process of producing a compound of formula (1) or (1-1) as defined above and wherein X is —NH—CH₂—, said process comprising the following step (iii-a):
(iii-a) converting a compound of formula (4) wherein R² and a are as defined for formula (1) or (1-1), respectively, or a salt thereof with an isocyanate or isothiocyanate of the following formula (5)

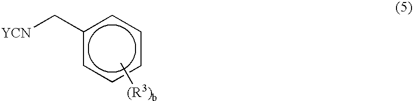

wherein X is —NH—CH₂— and wherein Y, R³, and b are as defined in formula (1) to said compound of formula (1).

Reaction (iii-a) may be performed in the presence of an auxiliary base, such as triethylamine or pyridine, wherein triethylamine is preferred. A typical reaction is performed for 14 hours, e.g. for 1.5-2 hours at a temperature of about 20° C.-40° C., preferably at about 25° C.-30° C.

The amide, cinnamoyl, alkinyl amide and alkoxyamide derivatives (wherein X is —CH₂—CH₂—, —CH═CH—, —C≡C—, or —C(R⁴)₂—O—) of the compounds of formula (1) or (1-1) as defined above may be prepared by a process comprising the following step (iii-b):
(iii-b) converting a compound of formula (4) wherein R² and a are as defined for formula (1) or (1-1), respectively, or a salt thereof with a compound of the following formula (8), or with a carbonic acid halide or an anhydride or an ester of a compound of formula (8)

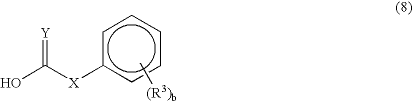

wherein X is selected from —CH₂—CH₂—, —CH═CH—, —C≡C—, or —C(R⁴)₂—O—, and wherein Y, R³, and b are as defined in formula (1) to said compound of formula (1) or (1-1).

The reaction (iii-b) may be performed by combining the compound of formula (8) and a compound of formula (4) in the presence of a condensing agent, such as carbodiimide or derivatives thereof like dicyclohexylcarbodiimide (DCC) or 1-ethyl-3-(3′-dimethylamino-propyl)-carbodiimide (EDC), N-hydroxysuccinimide derivatives or phosphoric acid derivatives such as diphenylphosphoryl azide (Carey and Sundberg, Advanced Organic Chemistry, Part B, 4^{th} Edition, 2001, Springer Science, p 172-178).

Alternatively, prior to the reaction (iii-b) the compound of formula (8) may be activated by converting it to the corresponding carbonic acid halide, preferably to the acid chloride, or by conversion to the anhydride or a reactive ester. The corresponding carbonic acid halide, the anhydride or ester of the compound of formula (8) can then be reacted with the compound of formula (4). The compounds of formula (8) can be converted to their acyl chlorides e.g. by the treatment with thionyl chloride, sulfonylchloride or phosphorus pentachloride. The conversion of the compounds of formula (8) to their anhydrides or to esters can be also performed according to the state of the art (Carey and Sundberg, Advanced Organic Chemistry, Part B, 4^{th} Edition, 2001, Springer Science, p 166-178).

The invention also provides a process of producing a compound of formula (1) or (1-1), wherein X is —CH₂—CH₂—, said process further comprising the following step (iii-c):
(iii-c) converting a compound of formula (4) wherein R² and a are as defined for formula (1) or (1-1), respectively, or the salt thereof with a compound of the following formula (9) or an acid halide, anhydride or ester thereof

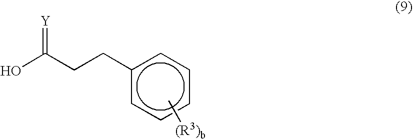

to a compound of formula (1) or (1-1), wherein Y, R³ and b are as defined in formula (1) further above.

Compounds of formula (9) may be prepared as described in WO 02/16318 using the Wittig-Homer reaction as shown in scheme 34 of WO 02/16318.

The invention also provides a process of producing a compound of formula (1) or (1-1), wherein X is —CH═CH—, said process further comprising the following step (iii-d):
(iii-d) converting a compound of formula (4) wherein R² and a are as defined for formula (1) or (1-1), respectively, or a salt thereof with a compound of the following formula (10) or an acid halide, ester or anhydride thereof

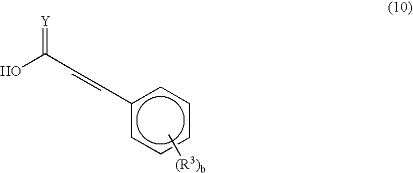

to a compound of formula (1) or (1-1), wherein Y, R³ and b are as defined in formula (1) further above.

Specifically, compounds of formula (1) or (1-1) wherein X is —CH═CH— may be prepared according to the following scheme:

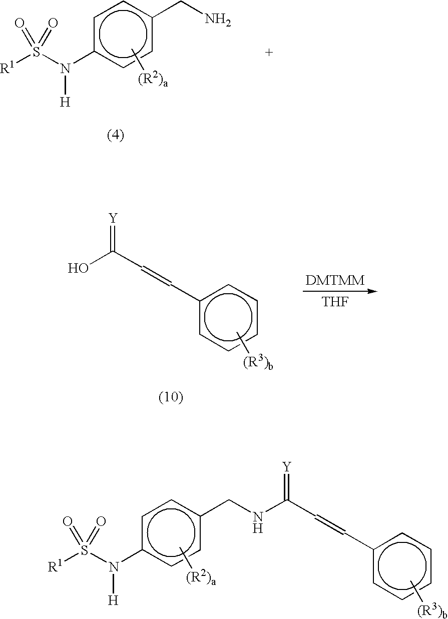

DMTMM is 4-(4,6-dimethoxy-1,3,5-triazin-2-yl)-4-methylmorpholinium chloride (Tetrahedron Lett., 1999, 40, 5327). This reaction may be performed in tetrahydrofuran (THF) as solvent. Alternatively, the amine component (4) and the cinnamic acid derivative (10) may be condensed using a carbodiimide such as EDC (1-ethyl-3-(3′-dimethylaminopropyl)-carbodiimide) as described in WO 2005/003084, notably with reference to scheme 1 and example 1-5 of WO 2005/003084. In a further alternative, the cinnamic acid derivative (10) may be condensed with the amine of formula (4) by activating the cinnamic acid derivatives (10) to the corresponding carbonic acid halide in an inert solvent followed by reacting the carbonic acid halide with the amine of formula (4), cf. scheme 34 of WO 02/16318. The cinnamic acid derivative (10) may be prepared from the corresponding benzaldehydes using the Wittig-Horner reaction e.g. as depicted in scheme 34 of WO 02/16318. The step of reducing the olefin to the corresponding saturated derivative of scheme 34 of WO 02/16318 will be left out.

The invention also provides a process of producing a compound of formula (1) or (1-1), wherein X is —C≡C—, said process further comprising the following step (iii-e):
(iii-e) converting a compound of formula (4) wherein R² and a are as defined for formula (1) or (1-1), respectively, or the salt thereof with a compound of the following formula (11) or an acid halide, ester or anhydride thereof

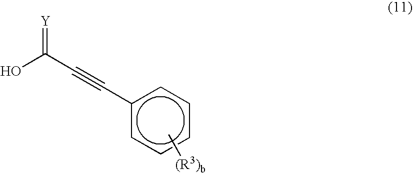

to a compound of formula (1) or (1-1) wherein Y, R³, and b are as defined in formula (1) further above, according to the following scheme:

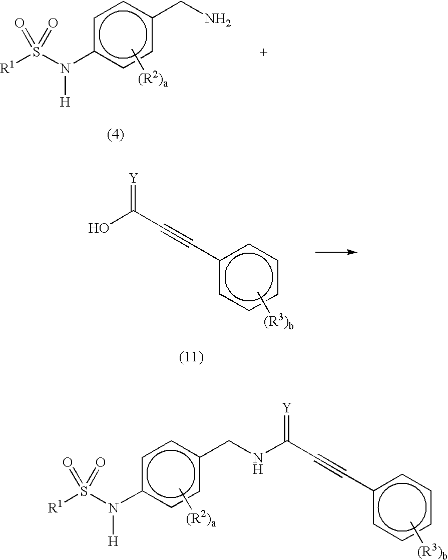

Compounds of formula (11) may be prepared by hydrolyzing a corresponding methyl ester for example using potassium carbonate in methanol. Reaction (iii-e) may be carried out as defined above for the case where X is —CH═CH—.

The invention also provides a process of producing a compound of formula (1-1), wherein X is —C(R⁴)₂—O—, said process further comprising the following step (iii-f):
(iii-f) converting a compound of formula (4) wherein R² and a are as defined for formula (1-1) or the salt thereof with a compound of the following formula (12) or an acid halide, ester or anhydride thereof

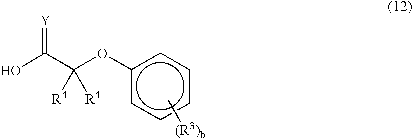

to a compound of formula (1) wherein Y, R³, R⁴ and b are as defined for formula (1-1).

The compounds of formula (1-1) wherein X is —C(R⁴)₂—O— may be prepared as described in WO 2006/051378, notably according to step 1E of scheme 1 of WO 2006/051378. Numerous specific examples are disclosed in WO 2006/051378.

The invention also provides a process of producing a compound of formula (1) or (1-1),

wherein X is —NH—CH₂— and Y is O, said process further comprising the following step (iii-f):
(iii-f) converting a compound of formula (4) wherein R² and a are as defined for formula (1) or (1-1), respectively, or the salt thereof with a compound of the following formula (13) to a compound of formula (1) or (1-1):

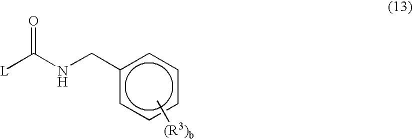

wherein L is a leaving group and R³ and b are as defined above. An example of group L is the phenoxy group (cf. example 9B). Other examples of L are phenoxy groups that are substituted in ortho or meta position by a halogen atom or a nitro group.

The present invention is further illustrated by the following examples that do not limit the scope of the present invention.

### EXAMPLE 1

### Production of N-(2-fluoro-4-cyanophenyl)methanesulfonamide

To 244 g (1.79 mol ) 3-fluoro-4-aminobenzonitrile in 1.8 l (22.3 mol) pyridine, 348 ml (4.49 mol) methanesulfonylchloride is added dropwise at a temperature of between 10° C. and 24° C.

The mixture is stirred for 30 min at 10° C. and then for further 16 hours without cooling. 3.3 l water is then added for hydrolysis of the dimesyl compound. The mixture is held under reflux for 1.5 hours, then cooled down to 0° C. and finally crystallized by stirring for 1 h at 0° C. The product is subsequently washed with each 300 ml water, 1 M hydrochloric acid and acetone and then dried for 12 hours at 50° C. in vacuo.

Yield: 355.4 g=92.6% of th.

Purity: HPLC: 99.9%

Identity determination:

MS: M+1 and fragmentation corresponds to expected structure

NMR: 1H and 13C signals can be interpreted according to the expected structure

Melting point: 202.2° C.

### EXAMPLE 2

### Step 2: Reduction of N-(2-fluoro-4-cyanophenyl)methanesulfonamide Using Pd/C

300 g (1.4 mol) N-(2-fluoro-4-cyanophenyl)methanesulfonamide are suspended in 600 ml methanol and 243 ml acetic acid. After the addition of 15 g 10% Pd/C, the hydrogenation is performed at 10° C. to 15° C. until 75 l hydrogen have been consumed. The catalyst is filtered off and the methanol is distilled off in a rotator. After the addition of 680 ml ethyl acetate, the solution is cooled to 0° C. The precipitate is washed twice with 250 ml ethyl acetate and then dried for 16 hrs at 50° C. in vacuum.

Yield: 331 g=84.95%

Purity: HPLC: 99.7%

Identity:

MS: M+1 and fragmentation corresponds to expected structure

NMR: 1H and 13C signals can be interpreted according to the expected structure

### EXAMPLE 3

### Step 2/Alternative Variant 1: Reduction of N-(2-fluoro-4-cyanophenyl)methanesulfonamide Using Raney-Nickel

200 g (0.934 mol) N-(2-fluoro-4-cyanophenyl)methanosulfonamide and 50 g Raney-nickel are dissolved in 2.2 l ethanol and 400 ml 25% ammonia solution and hydrogenated for 1.5 hrs at 95° C. and 5.5 bar. The autoclave is cooled to 60° C.

After the addition of 112 ml 20% sodium hydroxide, the mixture is stirred for additional 5 minutes and then filtered off. The solvent of the filtrate is removed and the remaining residue is dissolved in 1 l propanol-2 and 180 ml 25% hydrochloric acid at 50° C. After the addition of further 1.4 l propanol-2, the mixture is refluxed for 1 hr. After cooling to room temperature, the product is sucked off, washed twice with 300 ml propanol-2 and dried at 50° C. in vacuum for 16 hrs.

Yield: 177 g=74.35% of th.

Purity: HPLC: 92.1%

Identity

MS: M+1 and fragmentation corresponds to expected structure

NMR: 1H and 13C signals can be interpreted according to the expected structure

### EXAMPLE 4

### Step 2/Alternative Variant 2: Reduction of N-(2-fluoro-4-cyanophenyl)methanesulfonamide Using Lithium Aluminium Hydride

23.3 mmol N-(2-fluoro-4-cyanophenyl)methanesulfonamide is dissolved in 70 ml anhydrous THF under argon. At 0° C., 23.3 mmol lithium aluminium hydride is added in small portions, the mixture is then warmed up to reflux for 8 hours. The mixture is cooled down and 30 ml 2N hydrochloric acid is added dropwise. The precipitate is washed with 150 ml THF and the combined organic phases are dried under sodium sulfate. After washing with diethyl ether, the residue is dried in vacuo. The yield was 33 wt %.

### EXAMPLE 5

### Step 3: Production of SPM 14221

To 236 g (0.85 mol) 3-fluoro-4-(methanosutfonylamino)benzyl ammonium acetate, 500 ml DMF, 510 ml triethyamine and 800 ml ethylacetate are added. At 25 to 30° C., a solution of 150 g (0.73 mol ) 4-tert.-butylbenzylisothiocyanate and 1 l ethyl acetate is added dropwise within 1 hour. After stirring for 2 hrs at 30 ° C., the mixture is washed subsequently with 2 l of 12.5% hydrochloric acid and 900 ml water. The volume of the organic phase is reduced under reduced pressure to 1.3 l. After the addition of 1 l n-hexane, the product crystallizes. It is filtered off, washed with 240 ml n-hexane and dried at 40° C. in vacuo to a constant mass.

Yield: 257 g=82.97% of th.

Purity: HPLC: 99.5%

Identity:

MS: M+1 and fragmentation corresponds to expected structure

NMR: 1H and 13C signals can be interpreted according to the expected structure

### EXAMPLE 6

### Production, Isolation and Analysis of 1-tert-butyl-4-thiocyanomethylbenzene

450 g 4-t-butylbenzylbromid (1.98 mol) are dissolved in 2.2 l DMF. After the addition of 241 g (2.48 mol) potassium thiocyanate and 236 g (1.98 mol) potassium bromide, the mixture is heated to 130° C. and stirred at that temperature for 1.5 hrs. The mixture is then cooled down to room temperature and 2.25 l water and 1.15 l n-hexane is added. The organic phase containing the product is separated and washed with 400 ml water. n-hexane is then removed by rotary evaporation. The oily residue was dissolved in 320 ml acetonitrile and crystallized at −30° C. The product is filtered off, washed with acetonitrile and dried for 5 hrs in vacuo.

Yield: 255.3 g 62.77% of th.

Purity: HPLC: 98.2%

Identity:

MS: M+1 and fragmentation corresponds to expected structure

NMR: 1H and 13C signals can be interpreted according to the expected structure.

### EXAMPLE 7

### Preparation of N-(4-cyano-2-methylphenyl)-methanesulfonamide

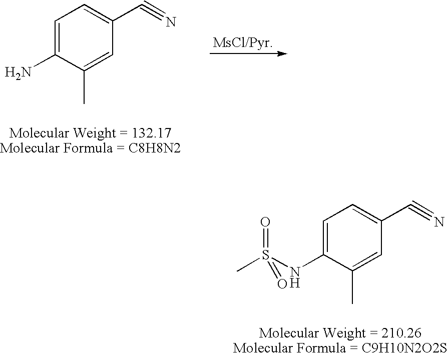

5 g (37.8 mmol ) 4-amino-3-methylbenzonitrile was dissolved in 38 ml (469 mmol) pyridine and the solution was cooled on ice to 15° C. Then, 7.3 ml (94 mmol) methanesulfonylchloride was slowly added dropwise. The temperature rose to 38° C. The solution was stirred for 72 hours at room temperature. Next, 63 ml water was added and the mixture was held under reflux for 10 minutes. After addition of 12 ml 5 N sodium hydroxide, the suspension became a clear solution. The mixture was held for 1 hour under reflux. Then, the mixture was neutralized by adding 60 ml 1 M hydrochloric acid and stirred for 1 hour at room temperature, whereupon the product precipitated. The product was filtered off and dried.

Yield: 7.7 g=91.6%

Analytical data:

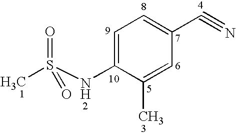

| H-NMR: | | | |
|---|---|---|---|
| 1 | CH₃SO₂— | 3.10 | ppm (S) |
| 2 | —NH | 9.47 | ppm (S) |
| 3 | PhCH₃ | 2.31 | ppm (S) |
| 5-10 | Ph-H | 7.47-7.69 | ppm (M) |
| C-NMR: | | | |
| 1 | CH₃SO₂— | 41.06 | ppm |
| 3 | PhCH₃ | 18.10 | |
| 4 | —CN | 119.13 | ppm |
| 5-10 | Ph | 107.49 | ppm |
| | | 123.61 | ppm |
| | | 131.15 | ppm |
| | | 132.91 | ppm |
| | | 134.84 | ppm |
| | | 140.94 | ppm |

MS: molecular ion: [M − H]⁻ = 209
fragmentation:

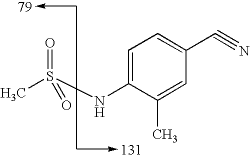

### EXAMPLE 8

### Preparation of 4-methanesulfonylamino-3-methyl-benzylammonium acetate

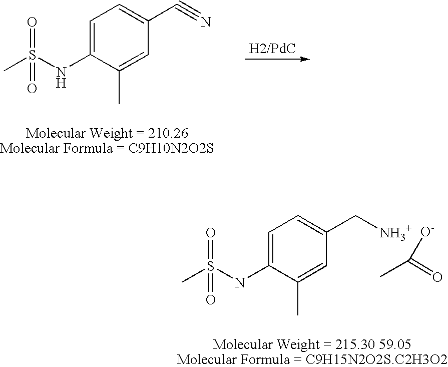

10 g (47.6 mmol) of N-(4-cyano-2-methylphenyl)-methanesulfonamide is suspended in 700 ml methanol. Thereto, a suspension of 1 g of 5% palladium on carbon catalyst in 20 ml glacial acetic acid is added. Hydrogenation is carried out at 5 bar for 12 hours. The maximum temperature is 23° C. 5.6 l of hydrogen are consumed.

After the reaction is completed, 1 g Celite is added and stirred for 30 minutes. The suspension is filtered over a D3 fritted-glass filter containing Celite. The solvent of the filtrate is removed under reduced pressure. The residue is dissolved in 150 ml toluene and the solvent is removed under reduced pressure. The residue is dissolved in 150 ml diethyl ether and the solvent is removed under reduced pressure. The residue is dissolved in 30 ml ethanol and 12 ml ethyl acetate is added. The solvent is removed under reduced pressure.

Yield: 10 g

Purity: 99.1%

### EXAMPLE 9

### Preparation of N-4-[3-(4-t-butylbenzyl)-ureidomethyl]-2-methylphenyl)methanesulfonamide

### A) Preparation of (4-t-butylbenzyl)-carbamic acid phenyl ester

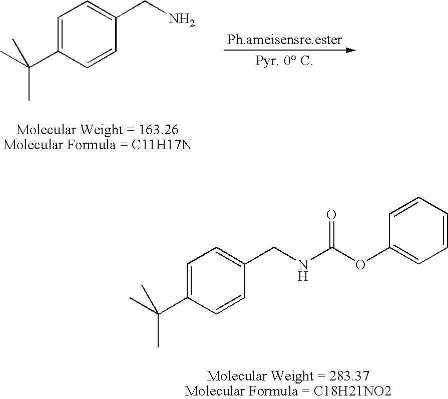

4.4 ml (25 mmol) of 4-t-butylbenzyl amine was added dropwise to 20 ml pyridine. The obtained solution was cooled on ice to 0° C. At this temperature, 3.2 ml (25 mmol) phenyl chloroformate was slowly added dropwise. The temperature of the solution rose to 10° C. The mixture was stirred overnight at room temperature. The mixture was diluted with 30 ml ethyl acetate and extracted with 30 ml 1 M hydrochloric acid and then with 30 ml 20% aqueous sodium chloride solution. The organic phase was dried over sodium sulfate, filtered and the solvent was evaporated.

Yield: 6.6 g reddish oil

Purity: 96.5%

### B) N-{4-[3-(4-t-butylbenzyl)-ureidomethyl]-2-methylphenyl}-methanesulfonamide

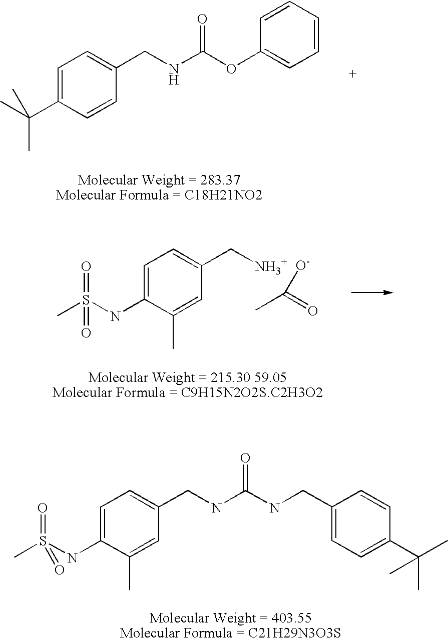

3 g (13.9 mmol) 4-methanesulfonylamino-3-methyl-benzylammonium acetate is suspended in 40 ml dichloromethane (DCM). To this mixture, 6 ml triethylamine is added. Then, a solution of 3 g (10.6 mmol) 4-t-butylbenzyl-carbamic acid phenyl ester in 40 ml DCM is added dropwise. The resulting mixture is stirred for 12 h at room temperature. Then, 12 ml acetonitrile is added and refluxed for 4 h, followed by stirring for 20 h at room temperature. The yellowish solution was diluted with 70 ml DCM and extracted three times each with 90 ml 1 M HCl and then with 20% aqueous sodium chloride. The organic phase was concentrated. The oily residue was recrystallized from a mixture of 10 ml DCM, 2 ml hexane and 1 ml diethyl ether.

Yield: 1 g

Purity: 93.3%

The present patent application claims the priority of European patent application 05 015 790.8, filed on Jul. 20, 2005, the content of which is incorporated herein by reference in its entirety.

1. A process of producing a compound of the following formula (3): 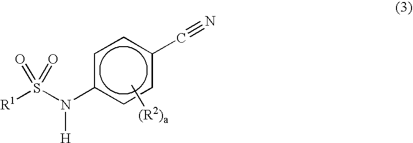
wherein
R¹ is a C₁₋₅ alkyl group,
R² is a halogen atom, a C₁₋₅ alkyl group, a C₂₋₅ alkenyl group, a C₂₋₅ alkynyl group, a halo C₁₋₅ alkyl group, a C₁₋₅ alkoxy group, a nitro group, or a hydroxy group, wherein multiple R² may be the same or may be different, and
a is an integer of from 0 to 4,
comprising reacting a compound of the following formula (2): 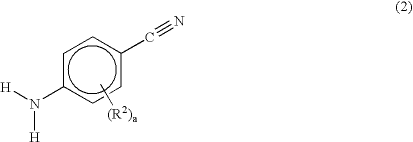
wherein R² and a are as defined above
with a C₁₋₅-alkanesulfonyl chloride or C₁₋₅-alkanesulfonic acid anhydride followed by hydrolyzing an N,N-disulfonated derivative of compound (3) to the compound of formula (3).

2. The process according to claim 1, wherein the compound of formula (2) is treated with more than one molar equivalent of C₁₋₅-alkanesulfonyl chloride or C₁₋₅-alkanesulfonic acid anhydride to produce a reaction mixture containing a disulfonated product of the following formula (3a): 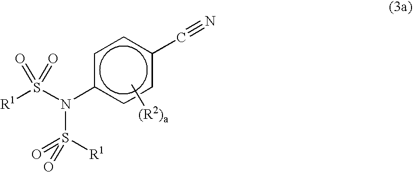
followed by hydrolyzing the compound of formula (3a) to the compound of formula (3) in an aqueous solvent.

3. The process according to claim 1, wherein said hydrolyzing is performed by heating in an aqueous solution of a base.

4. The process according to claim 1, wherein R² is methyl, ethyl, vinyl, ethynyl, fluoro, chloro, bromo, iodo, or nitro; and a is 1 or 2.

5. The process according to, wherein R¹ is methyl or ethyl; R² is fluoro; and a is 1 or 2.

6. A process or producing a compound of the following formula (1): 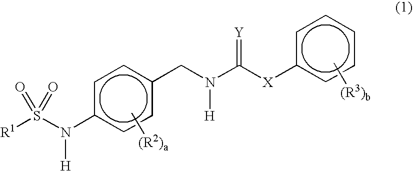
wherein
X is —NH—CH₂—, —CH₂—CH₂—, —CH═CH—, or —C≡C—,
Y is O or S,
R¹ is a C₁₋₅ alkyl group,
R² is a halogen atom, a C₁₋₅ alkyl group, a C₂₋₅ alkenyl group, a C₂₋₅ alkynyl group, a halo C₁₋₅ alkyl group, a C₁₋₅ alkoxy group, a nitro group, or a hydroxy group, wherein multiple R² may be the same or may be different, and
R³ is a halogen atom, a C₁₋₆ alkyl group, a halo C₁₋₆ alkyl group, a C₁₋₅ alkoxy group, a C₁₋₅ alkylthio group, a nitro group, a C₁₋₅ alkoxy C₁₋₅ alkoxy group, a C₁₋₅ alkoxy C₁₋₅ alkyl group, a C₁₋₅ alkoxy C₁₋₅ alkoxy C₁₋₅ alkyl group, C₁₋₅ alkylsulfonyl group, C₁₋₅ alkylcarbonyl group, C₁₋₅ alkoxycarbonyl group, C₁₋₅ alkoxycarbonyl C₁₋₅ alkoxy group, a C₁₋₅ alkoxy C₁₋₅ alkylamino group, morpholino, wherein multiple R³ may be the same or may be different,
a is an integer of from 0 to 4, and
b is an integer of from 0 to 5,
said process comprising the following step (i):
(i) converting a compound of the following formula (2) 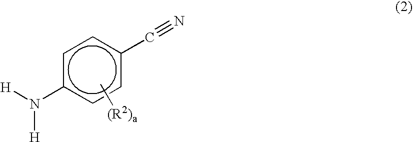
wherein R² and a are as described for formula (1) to a compound of the following formula (3): 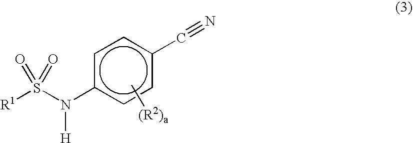
wherein R¹, R² and a are as described for formula (1) by reacting a compound of formula (2) with a C₁₋₅-alkanesulfonylchloride or C₁₋₅-alkanesulfonic acid anhydride followed by hydrolyzing an N,N-disulfonated derivative of compound (3) to the compound of formula (3).

7. A process or producing a compound of the following formula (1-1): 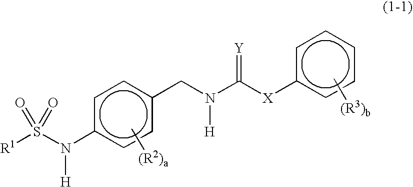
wherein
X is —NH—CH₂—, —CH₂—CH₂—, —CH═CH—, —C≡C— or —C(R⁴)₂—O—,
Y is O or S,
R¹ is a C₁₋₅ alkyl group,
R² is a halogen atom, a C₁₋₅ alkyl group, a C₁₋₅ alkoxy group, a nitro group, or a hydroxy group, wherein multiple R² may be the same or may be different, and
R³ is a halogen atom, a C₁₋₆ alkyl group, a halo C₁₋₆ alkyl group, a C₁₋₅ alkoxy group, a C₁₋₅ alkylthio group, a nitro group, a C₁₋₅ alkoxy C₁₋₅ alkoxy group, a C₁₋₅ alkoxy C₁₋₅ alkyl group, a C₁₋₅ alkoxy C₁₋₅ alkoxy C₁₋₅ alkyl group, C₁₋₅ alkylsulfonyl group, C₁₋₅ alkylcarbonyl group, C₁₋₅ alkoxycarbonyl group, C₁₋₅ alkoxycarbonyl C₁₋₅ alkoxy group, a C₁₋₅ alkoxy C₁₋₅ alkylamino group, morpholino, wherein multiple R³ may be the same or may be different,
R⁴ is hydrogen, a C₁₋₅ alkyl group, or halo, whereby multiple R⁴ may be the same or may be different,
a is an integer of from 0 to 4, and
b is an integer of from 0 to 5,
said process comprising the following step (i):
(i) converting a compound of the following formula (2) 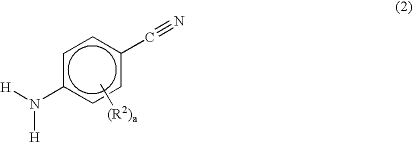
wherein R² and a are as described for formula (1-1) to a compound of the following formula (3): 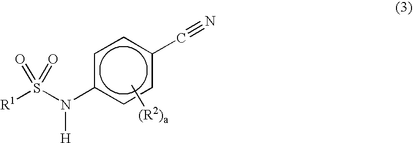
wherein R¹, R² and a are as described for formula (1-1) by reacting a compound of formula (2) with a C₁₋₅-alkanesulfonylchloride or C₁₋₅-alkanesulfonic acid anhydride followed by hydrolyzing an N,N-disulfonated derivative of compound (3) to the compound of formula (3).

8. The process according to claim 6, said process comprising the following step (ii):
(ii) converting a compound of formula (3) wherein R² and a are as described for formula (1) to a compound of the following formula (4) or a salt thereof:

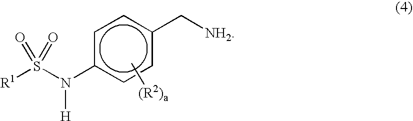

9. The process according to claim 7, said process comprising the following step (ii):
(ii) converting a compound of formula (3) wherein R² and a are as described for formula (1-1) to a compound of the following formula (4) or a salt thereof:

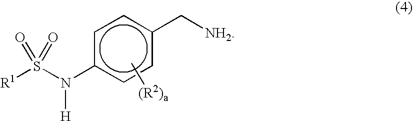

10. The process according to claim 8, wherein step (ii) is performed in acetic acid using hydrogen as a reducing agent and palladium on carbon as a catalyst.

11. The process according to claim 8, comprising the following step (iii-a):
(iii-a) converting a compound of formula (4) wherein R² and a are as defined for formula (1) or (1-1), respectively, or the salt thereof with an isocyanate or isothiocyanate of the following formula (5) to a compound of formula (1) or (1-1): 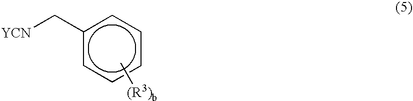
wherein X is —NH—CH₂—;
Y is O or S:
R³ is a halogen atom, a C₁₋₆ alkyl group, a halo C₁₋₆ alkyl group, a C₁₋₅ alkoxy group, a C₁₋₅ alkylthio group, a nitro group, a C₁₋₅ alkoxy C₁₋₅ alkoxy group, a C₁₋₅ alkoxy C₁₋₅ alkyl group, a C₁₋₅ alkoxy C₁₋₅ alkoxy C₁₋₅ alkyl group, C₁₋₅ alkylsulfonyl group, C₁₋₅ alkylcarbonyl group, C₁₋₅ alkoxycarbonyl group, C₁₋₅ alkoxycarbonyl C₁₋₅ alkoxy group, a C₁₋₅ alkoxy C₁₋₅ alkylamino group, morpholino, wherein multiple R³ may be the same or may be different; and
b is an integer of from 0 to 5.

12. The process according to claim 11, wherein Y is S and wherein the compound of formula (5) is produced by reacting a compound of the following formula (6): 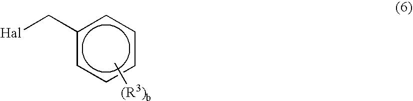
wherein HaI is a halogen atom: R³ is a halogen atom, a C₁₋₆ alkyl group, a halo C₁₋₆ alkyl group, a C₁₋₅ alkoxy group, a C₁₋₅ alkylthio group, a nitro group, a C₁₋₅ alkoxy C₁₋₅ alkoxy group, a C₁₋₅ alkoxy C₁₋₅ alkyl group, a C₁₋₅ alkoxy C₁₋₅ alkoxy C₁₋₅ alkyl group, C₁₋₅ alkylsulfonyl group, C₁₋₅ alkylcarbonyl group, C₁₋₅ alkoxycarbonyl group, C₁₋₅ alkoxycarbonyl C₁₋₅ alkoxy group, a C₁₋₅ alkoxy C₁₋₅ alkylamino group, morpholino, wherein multiple R³ may be the same or may be different; and b is an integer of from 0 to 5
with rhodanide and converting the resulting thiocyanate to an isothiocyanate of formula (5).

13. The process according to claim 8, comprising the following step (iii-b):
(iii-b) converting a compound of formula (4) wherein R² and a are as defined for formula (1) or (1-1), respectively, or a salt thereof with a compound of the following formula (8) or an acid halide, ester or anhydride thereof, 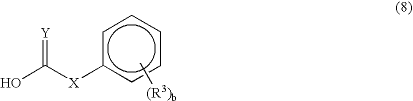
to a compound of formula (1) or (1-1), wherein Y, R³, and b are as defined in claim 6 and wherein X is —CH₂—CH₂—, —CH═CH—, —C≡C—, or —C(R⁴)₂—O—.

14. The process according to claim 6, wherein R¹ is methyl or ethyl; R² is methyl, ethyl, fluoro, chloro, bromo, iodo, or nitro; and a is 1 or 2.

15. The process according to claim 6, wherein R¹ is methyl or ethyl; R² is a fluorine or chlorine atom; a is 1 or 2; R³ is t-butyl or i-propyl; and b is 1.

16. The process according to claim 6, wherein b is an integer of from 1 to 3 and at least one R³ is an optionally halogenated t-butyl or i-propyl in para position to group X.

17. The process according to claim 6, wherein Y is O.

18. A process of producing an isothiocyanate compound of formula (5) as defined in claim 11, comprising converting a compound of the following formula (6): 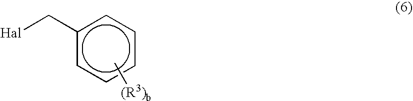
wherein HaI is a halogen atom; R³ is a halogen atom, a C₁₋₆ alkyl group, a halo C₁₋₆ alkyl group, a C₁₋₅ alkoxy group, a C₁₋₅ alkylthio group, a nitro group a C₁₋₅ alkoxy C₁₋₅ alkoxy group, a C₁₋₅ alkoxy C₁₋₅ alkyl group, a C₁₋₅ alkoxy C₁₋₅ alkoxy C₁₋₅ alkyl group, C₁₋₅ alkylsulfonyl group, C₁₋₅ alkylcarbonyl group, C₁₋₅ alkoxycarbonyl group, C₁₋₅ alkoxycarbonyl C₁₋₅ alkoxy group, a C₁₋₅ alkoxy C₁₋₅ alkylamino group, morpholino, wherein multiple R³ may be the same or may be different: and b is an integer of from 0 to
with rhodanide to a thiocyanate of the following formula (7): 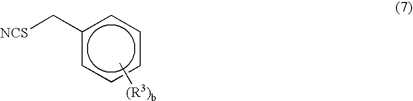
followed by converting the thiocyanate of formula (7) to an isothiocyanate of formula (5).

19. The process according to claim 18, wherein the thiocyanate of formula (7) is converted to an isothiocyanate of formula (5) by heating for 1 to 3 hours to 120 to 150° C. in a solvent.

20. A process of producing a compound of formula (1) as defined in claim 6, comprising the reduction of a compound of formula (3) to a compound of formula (4) or a salt thereof in acetic acid using palladium on carbon as the catalyst in the presence of hydrogen.

21. A process of producing a compound of formula (1-1) as defined in claim 7, comprising the reduction of a compound of formula (3) to a compound of formula (4) or a salt thereof in acetic acid using palladium on carbon as the catalyst in the presence of hydrogen.

22. The compound of the following formula (7): 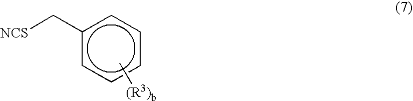
wherein R³ and b are as defined in claim 6, preferably R³ is a C₁₋₆ alkyl group or a halogen atom and b is an integer of from 0 to 5.

23. (canceled)

24. The process of claim 19 wherein wherein the thiocyanate of formula (7) is converted to an isothiocyanate of formula (5) in the presence of ZnCl2 or KBr.
